(19) United States
(12) Patent Application Publication
(10) Pub. No.: US 2025/0347680 A1
(43) Pub. Date: Nov. 13, 2025

(54) METHODS OF OPTIMIZING KINASE INHIBITORS

(71) Applicant: Brandeis University, Waltham, MA (US)

(72) Inventors: Niels Bradshaw, Lincoln, MA (US); Emily Stadnicki, Watertown, MA (US)

(73) Assignee: Brandeis University, Waltham, MA (US)

(21) Appl. No.: 19/202,514

(22) Filed: May 8, 2025

Related U.S. Application Data
(60) Provisional application No. 63/644,812, filed on May 9, 2024.

(51) Int. Cl. G01N 33/50; C12Q 1/42; C12Q 1/48

(52) U.S. Cl. CPC G01N 33/5038; C12Q 1/42; C12Q 1/485; G01N 2333/912; G01N 2333/916; G01N 2500/02

(57) ABSTRACT

Described herein is a method of determining if a kinase inhibitor is a phosphatase enhancer or inhibitor, the method including providing a phosphorylated target kinase substrate; contacting the kinase inhibitor, the phosphorylated target kinase substrate and the phosphatase under conditions for dephosphorylation of the target kinase substrate by the phosphatase; quantitating dephosphorylation of the phosphorylated target kinase substrate in the presence and absence of the kinase inhibitor; and determining the kinase inhibitor is a phosphatase enhancer when its presence increases the rate of dephosphorylation of the target kinase, or determining the kinase inhibitor is a phosphatase inhibitor when its presence decreases the rate of dephosphorylation of the target kinase.

### CROSS-REFERENCE TO RELATED APPLICATIONS

This application claims priority to U.S. Provisional Application 63/644,812 filed on May 9, 2024, which is incorporated herein by reference in its entirety.

### SEQUENCE LISTING

The Instant Application contains a Sequence Listing which has been submitted electronically in XML format and is hereby incorporated by reference in its entirety. Said XML copy, created on May 5, 2025, is named “SEQ_LIST—107648063.xml” and is 3,645 bytes in size. The Sequence Listing does not go beyond the disclosure in the application as filed.

### FIELD OF THE DISCLOSURE

The present disclosure is related to methods of identifying kinase inhibitors, particularly kinase inhibitors with improved potency and specificity.

### BACKGROUND

The human genome contains over 500 protein kinases. These kinases affect intracellular signal transduction pathways through protein phosphorylation. Aberrant kinase activity has been implicated in numerous diseases, leading to an intense drug discovery effort to develop efficacious anti-kinase therapeutics, resulting in over 20 FDA approved targeted kinase inhibitors mainly for the treatment of cancers including chronic myeloid leukemia and non-small cell lung cancer. While these efforts have revolutionized cancer therapy, a large degree of active site conservation throughout the kinase family causes most kinase inhibitors to possess promiscuous inhibition activities towards many kinases. While often needed for a complete response, this polypharmacology can also lead to side effects that negatively affect the quality of life, largely preventing kinase inhibitors from becoming therapeutics for chronic non-lethal diseases such as rheumatoid arthritis, where selectivity becomes a much larger requirement.

Kinase inhibitors are also common chemical probes to elucidate the role of a kinase or signaling pathways in cellular processes or disease. These fundamental studies are frequently confounded by off-target kinase inhibition affecting unintended signaling pathways.

Four p38 mitogen-activated protein kinase enzymes (p38 MAPK) isoforms (alpha, beta, gamma and delta respectively) have been identified, each displayingdifferent patterns of tissue expression. p38 MAP kinase is believed to play a pivotal role in many of the signaling pathways that are involved in initiating and maintaining chronic, persistent inflammation in human disease. p38α MAP kinase is a critical regulatory node for the DNA damage response and for inflammatory pathways. The activation loop of p38α is dual-phosphorylated on a threonine and tyrosine, with threonine phosphorylation causing a 10,000-fold increase in kinase activity. In the cell, p38α phosphorylation is controlled by upstream kinases, autophosphorylation, and a suite of protein phosphatases. Because p38α activation contributes to inflammatory diseases including myocardial ischemia and neurodegeneration, diverse p38α specific kinase inhibitors have been identified and studied in the clinic. Small-molecule-competitive kinase inhibitors have achieved remarkable clinical success, often achieving target specificity by binding to inactive kinase conformations (type II inhibitors). However, a downside to many kinase inhibitors is their lack of specificity and general potency, causing drug side effects due to off target effects.

What is needed are novel methods for identifying successful kinase inhibitors such as p38α inhibitors, particularly inhibitors with greater potency and specificity.

### BRIEF SUMMARY

In an aspect, described herein is a method of determining if a kinase inhibitor is a phosphatase enhancer or inhibitor, the method including providing a phosphorylated target kinase substrate; contacting the kinase inhibitor, the phosphorylated target kinase substrate and the phosphatase under conditions for dephosphorylation of the target kinase substrate by the phosphatase; quantitating dephosphorylation of the phosphorylated target kinase substrate in the presence and absence of the kinase inhibitor; and determining the kinase inhibitor is a phosphatase enhancer when its presence increases the rate of dephosphorylation of the target kinase substrate, or determining the kinase inhibitor is a phosphatase inhibitor when its presence decreases the rate of dephosphorylation of the target kinase substrate.

### BRIEF DESCRIPTION OF THE DRAWINGS

FIGS. 1A-C show type II p38 inhibitors modulate dephosphorylation of p38α by WP1 phosphatase. 1A shows a schematic describing the mechanism pf type II p38α inhibitors causing a change in activation loop conformation. This is followed by a change in phosphatase activity as a result of change in phosphothreonine accessibility. Phosphorylation sites pT180 and pT182 are depicted as spheres and sticks on the activation loop cartoon. 1B top panel shows single turnover kinetics of dephosphorylation pf p38α (0.25 μM) phosphothreonine by WP1 (2.5 μM) in the presence of excess p38 inhibitors (1.25 μM). Data are fit to an exponential decay (k_{obs} displayed were averaged from an n=3±1SD). 1B bottom panel shows the fold change in k_{obs} relative to a DMSO treated control (error displayed is error of the fit propagated to DMSO error of the fit). 1C shows chemical structures of inhibitors that had the largest impact on phosphatase activity.

FIGS. 2A-B show multiple turnover conditions do not affect the indirect effect of compounds on WIP1 catalyzed p38α dephosphorylation. 2A shows multiple turnover kinetics of fluorescein diphosphate (50 μM) hydrolysis by WP1 (30 nM in the presence of 1.25 μM inhibitors) showing no change in WP1 activity toward a generic substrate with the addition of p38α inhibitors. 2B shows multiple turnover reactions of WP1 (0.25 μM) dephosphorylation of p38α-2p (20 UM on left panel and displayed on right x-axis) showing similar inhibitor effect compared to single turnover reactions (FIG. 1B).

FIGS. 3A-D show WP1 stimulating p38α inhibitors induce a unique activation loop confirmation, presenting phosphothreonine for dephosphorylation. 3A shows an overlay of X-ray crystal structure of human apo p38-2p and pexmetinib bound human p38-2p emphasizing activation loop and phosphorylation site rearrangement. Left zoom shows pexmetinib induced A-loop confirmation and coordination of phosphotyrosine. Note that the phosphothreonine is unresolved, likely due to flexibility. Right zoom shows apo p38-2p A-loop and coordination of phosphor-tyrosine and phosphor-threonine. 3B shows the chemical structure of pexmetinib and DP802. 3C shows p38-p2 bound to DP802 overlayed with p38-2p bound to pexmetinib. Phosphotyrosine coordination and DFG (168-170) motif is highlighted. 3D shows p38-2p bound to pexmetinib, nilotinib, and BIRB 796 overlay. Phosphotyrosine coordination and DFG (168-170) motif is highlighted.

FIGS. 4A-C show ordering the aD helix is not sufficient to increase WIP1 activity. 4A shows an overlay of X-ray crystal structures of p38α-2p bound pexmetinib, nilotinib, and BIRB 796 highlighting the N to C lobe linker, including the aD helix (109-123) in a ribbon model showing the varied level of resolution in the region. 4B shows single turnover kinetics of dephosphorylation of p38α (0.25 μM) phosphor-threonine by WP1 (2.5 μM) in the presence of an excess of inhibitors (1.25 μM) with and without MKK3b KM peptide (1.24 μM). Data are fit to an exponential decay with the time being shown on a log x-axis (n=2, error displayed is error of the fit). 4C shows 2Fo-Fc electron density (contoured at 1 σ) of p38α bound to BIRB 796 (left) and nilotinib (right) showing a disulfide bond between C119 and C162.

FIGS. 5A and B illustrate a global shift of N lobe and P-loop occur within compound bound structures and within apo structure variants. 5A shows an overlay of X-ray crystal structures of p38α-2p bound pexmetinib, nilotinib and BIRB 796 highlighting the P-loop (29-36) in ribbons and F169 and V30 which are coordinate in pexmetinib and nilotinib bound structures. 5B shows an overlay of X-ray crystal structures of apo p38α, chain A and chain B of human p38α and chain A of the mouse apo p38α (PDB: 3PY3) highlighting the shift of the n lobe and P-loop indicating different confirmational sampling.

FIG. 6 shows an overlay of X-ray crystal structures of p38α bound nilotinib with p38β-Op bound nilotinib (PDB: 3GP0), highlighting the activation loop (167-184) in ribbons and D168, F169 and Y182 shown in sticks to emphasize the conserved activation loop flip with the addition of nilotinib.

FIGS. 7A-D illustrate that tyrosine phosphorylation is not required for stimulation of WIP1. 7A shows WIP1 (2.5 μM) dephosphorylation kinetics of single threonine phosphorylated p38α: Y182F (0.25 UM) in the presence of excess p38α inhibitors (5-fold molar excess). Data were fit to an exponential decay (n=2±error of the fit). 7B shows an X-ray crystal structure of unphosphorylated p38α bound to pexmetinib overlayed with dual phosphorylated p38α bound to pexmetinib showing a similar activation loop conformation. DFG (168-170), Y182 and R 186 are shown as sticks. 7C shows fold change comparisons of p38 (0.25 μM) phosphorylation rates with SAP (4× molar excess), PPM1A (0.5 μM), DUSP3 (50 μM), all unique phosphatases that can target p38α, in the presence of excess inhibitor (5-fold excess) (error displayed is error of the fit propagated to DMSO error of the fit). 7D shows a fold change comparison of rate of DUSP3 (15 μM) dephosphorylation of Y182 (T180A mutant (0.25 μM) in the presence of excess inhibitor (5-fold excess) (n=2±error of the fit).

FIGS. 8A-F show type II inhibitors control p38 phosphorylation in cell. 8A is a scheme describing the model that activation loop left inhibitors will cause a decrease in pT and 2p p38 in cells along with a prediction that the compounds, which decrease phosphatase activity towards p38α would cause an accumulation of pT and 2P-p38α. 8B shows a western blot of HL60 cells treated with 10 μM inhibitor (100 UM for imatinib due to low potency) for 30 minutes after a 30 minute stimulation of the inflammatory response with 40 ng/mL TNFα. Lysate was blotted for various phosphorylation levels of p38α. Actin serves as a loading control. 8C shows p38 activity was measured using a readout of TNFα after LPS stimulation and treatment with inhibitors at the same concentrations as in panel B. 8D shows a correlation plot of quantified p38-2p western blot and vs. WIP1 k_{obs} values as shown in FIG. 1B. 8E shows cellular IC50 values of nilotinib, BIRB 796 and LY S2228820 measured using TNFα output as a readout of p38α activity. 8F shows a western blot of varying concentrations of inhibitors showing the effect of p38α phosphorylation level occurs at IC50 levels of inhibitor treatment.

FIG. 9 shows a western blot of HL60 cells treated with 10 UM inhibitor (100 μM for imatinib due to low potency) for 30 minutes after a 60 minute stimulation of the inflammatory response with lipopolysaccharide (LPS (1× concentration). Lysate was blotted for various phosphorylation levels of p38α. Actin serves as a loading control.

The above-described and other features will be appreciated and understood by those skilled in the art from the following detailed description, drawings, and appended claims.

### DETAILED DESCRIPTION

Described herein is the discovery that kinase inhibitors can interact with protein phosphatases, either positively, by enhancing phosphatase driven inactivation of the target kinase, or negatively, by inhibiting kinase dephosphorylation. The data presented herein suggests that these effects occur in a cellular context, and it is hypothesized that they impact inhibitor potency and pharmacokinetics. Described herein is a new strategy for assaying and refining kinase inhibition that includes how the inhibitor interacts with phosphatases. A general method for profiling kinase inhibitor/phosphatase interactions for drug optimization is provided.

In an aspect, to take advantage of phosphatase synergy, a cycle of optimization can be added to traditional kinase inhibitor screening in which lead compounds are assayed for changes in activity of a physiologically relevant phosphatase or phosphatases. To validate this method, a library of known kinase inhibitors was screened against p38α MAP kinase and it was discovered that a subset of commercially available p38α inhibitors either enhance or inhibit p38 dephosphorylation by the PPM family phosphatases PPMIA and WIPI. Advantageously, the methods described herein can be applied as a screening step in existing pipelines to identify kinase inhibitors that could have higher specificity and potency because of their synergistic effects with native cellular phosphatases. Drugs that synergize with native phosphatases are more specific because phosphatases themselves are cell type specific. They would have increased potency because of a dual mechanism of inhibition through dephosphorylation of the target kinase.

More specifically, threonine dephosphorylation of p38α MAP kinase is controlled by WIP1, PPM1A, PP2A, and DUSP phosphatases, while DUSP and PTP phosphatases additionally dephosphorylate p38α phosphotyrosine. Using a set of type II inhibitors targeting p38α MAPK inase, the inventors discovered inhibitors that trap activation-loop conformations that modulate phosphatase recognition in biochemically reconstituted reactions and in human cells. X-ray crystal structures of inhibitor bound p38α reveal a flipped conformation of the activation loop that presents the activation loop phosphothreonine for dephosphorylation that explains increased dephosphorylation rate. human PPM phosphatases, including WIP1, are similarly responsive to substrate conformation.

As used herein, kinases transfer phosphate groups from high-energy donor molecules, such as ATP, to specific target proteins (substrates), a process called phosphorylation, that ultimately leads to the altered biological function of the target protein. Kinases phosphorylate a serine, threonine, or tyrosine residue in a target protein.

Phosphatases remove a phosphate group from a target protein.

The target protein itself may be a kinase.

In an aspect, a method of determining if a kinase inhibitor is a phosphatase enhancer or inhibitor comprises providing a phosphorylated target kinase substrate; contacting the kinase inhibitor, the phosphorylated target kinase substrate and the phosphatase under conditions for dephosphorylation of the target kinase substrate by the phosphatase; quantitating dephosphorylation of the phosphorylated target kinase substrate in the presence and absence of the kinase inhibitor; and determining the kinase inhibitor is a phosphatase enhancer when its presence increases the rate of dephosphorylation of the target kinase substrate, or determining the inhibitor is a phosphatase inhibitor when its presence decreases the rate of dephosphorylation of the target kinase substrate.

An exemplary kinase inhibitor for use in the methods described herein is an ATP-competitive kinase inhibitor, such as a type I or type II inhibitor. A type I inhibitor targets the ATP binding pocket of the kinase in its active state. Type II inhibitors bind the ATP binding pocket of the kinase in its inactive state. Because they bind to the ATP binding pocket, the ATP-competitive kinase inhibitors compete with ATP.

In another aspect, the kinase inhibitor is an allosteric kinase inhibitor. As used herein, an allosteric kinase inhibitor is an inhibitor that targets an allosteric pocket other than the ATP pocket. Trametinib, for example, is an allosteric inhibitor of the MEK1 and MEK2 kinases which binds adjacent to the ATP pocket. DP802 is a p38α inhibitor which binds to Arg70, which is outside of the ATP hinge region.

Exemplary target kinases for the methods described herein include the serine/threonine protein kinases such as the mitogen-activated protein kinases (MAP kinases), the tyrosine-specific kinases (platelet-derived growth factor receptor (PDGFR), epidermal growth factor receptor (EGFR), insulin receptor, insulin-like growth factor 1 receptor (IGF1R) and stem cell factor receptor (c-kit)), the receptor tyrosine kinases (epidermal growth factor receptor family, fibroblast growth factor receptor family, vascular endothelial growth factor receptor family, RET receptor family, ephrin receptor family, discoidin receptor domain receptor family), the receptor-associated tyrosine kinases (J anus kinases), and the dual-specificity kinases (MEK (MAPKK)).

In an aspect, the target kinase is a MAP kinase such as the extracellular signal-regulated kinases (M apK1 (ERK2), MAPK3 (ERK1)), the c-Jun N-terminal kinases (JNK1, JNK2, JNK3), the p38 isoforms (MAPK11 (p38-beta), MAPK12 (p38-gamma), MAPK13 (p-38 delta), MAPK14 (p38-alpha)), ERK5(MAPK7), ERK3(MAPK6), ERK4(MAPK4), and ERK7/8 (MAPK15).

In the method, the target kinase is phosphorylated to provide a phosphorylated target kinase substrate. Methods of phosphorylating target kinases in vitro using a cognate upstream kinase for the targeted kinase and ATP are well-known in the art. As used herein, a cognate upstream kinase is a kinase in the same signaling cascade as the target kinase with activity that precedes that of the target kinase in the cascade.

In an aspect, when the target kinase is a part of the inflammatory and DNA damage response such as p38α, phosphorylation can be initiated by TNFα, LPS, initiating the DNA damage response through UV-mediated DNA damage, H202-mediated oxidative stress, or pH stress via addition of a high pH buffer.

Exemplary phosphatases for the methods described herein are phosphatases that can dephosphorylate the target kinase. Phosphatases can be classified as follows: PPPs (phosphoprotein phosphatases), PPM s (metal-dependent protein phosphatases) and PTPs (protein tyrosine phosphatases). PPPs and PPM s dephosphorylate phosphoserine and phosphothreonine residues, whereas the PTPs dephosphorylate phosphotyrosine amino acids. For a given target kinase, one of ordinary skill in the art can identify phosphatases that dephosphorylate a target kinase.

For p38α. MAPK inase, phosphatases include WIP1, PPM1A, PP2A, PTP, and DUSP phosphatases.

Exemplary phosphatase/kinase pairs include ERK/JNK, PPM1A/AKT PP2A/AKT, PP2A/ERK, DUSPs/JNK, and WIP1/ATM.

Conditions for dephosphorylation of the target kinase substrate by the phosphatase can be readily determine by one of ordinary skill in the art for the particular target kinase/phosphatase pair.

The method also includes quantitating dephosphorylation of the phosphorylated target kinase substrate in the presence and absence of the ATP-competitive kinase inhibitor. Exemplary methods include utilizing a phosphorylated kinase including radioactive phosphate groups and then measuring radioactive phosphate release over time. Fluorescent probes such as 6,8-difluoro-4-methylumbelliferyl phosphate (DiFMUP) can be used as an alternative to radiolabeled phosphate.

Based on the quantitation, it is determined that the kinase inhibitor is a phosphatase enhancer when its presence increases the rate of dephosphorylation of the target kinase, or the kinase inhibitor is a phosphatase inhibitor when its presence decreases the rate of dephosphorylation of the target kinase.

Validation that the kinase inhibitor is a phosphatase enhancer/inhibitor can be done by different methods. In an aspect, the method can further include determining the x-ray crystal structure of the kinase inhibitor bound to the phosphorylated target kinase substrate, and identifying changes in the phosphorylated target kinase substrate in the presence of the bound kinase inhibitor. In an aspect, the change in the phosphorylated target kinase substrate in the presence of the bound kinase inhibitor is a change in the activation loop conformation.

In other aspect, the method can further include a cellular assay for monitoring the effect on the target kinase phosphorylation, a cellular assay for monitoring enhancement/inhibition of phosphatase activity, and the like.

In an aspect, the kinase inhibitor is a member of a library. Libraries of kinase inhibitors may be screened for binding affinity to the target kinase and/or target kinase inhibition through a high throughput kinase assay measuring the uptake of ATP. The kinase inhibitors identified from library screens can then me used in the optimization methods described herein.

In an aspect, the method further comprises administering the kinase inhibitor to a subject in need of such treatment. Exemplary conditions for treatment with kinase inhibitors include cancer, neurodegenerative disease, and inflammatory disease.

In an aspect, when the kinase inhibitor is identified as a phosphatase enhancer, the method can further comprise administering the kinase inhibitor to a subject in need of treatment with a kinase inhibitor.

Also included herein is a multiwell assay, wherein each well comprises a phosphorylated target kinase substrate, a phosphatase, a buffer, and a means for quantitating dephosphorylation of the phosphorylated target kinase substrate. In an aspect, the means for quantitating dephosphorylation of the phosphorylated target kinase substrate comprises a reagent. In another aspect, the means for quantitating dephosphorylation of the phosphorylated target kinase substrate comprises a radioactive or fluorescent phosphate probe.

The invention is further illustrated by the following non-limiting examples

### Examples

### Methods

Protein expression constructs: Full length PPM1A, p38, and DUSP3 were generated by isolation of their respective coding sequences from HEK293 genomic DNA and inserted into pET 47b vectors with a 6-His tag. Mutations were introduced to the p38 expression construct using the QuikChange® site-directed mutagenesis kit. pCDFDuet-MKK6-EE was a gift from Kevin Janes (Addgene plasmid #82718; RRID: Addgene_82718). The cloning sequence was inserted into a pET 47b vector with a 6-His tag. WIP1-genescript.

Protein expression and purification: All proteins were expressed in E. coli BL21 (DE3) cells were grown at 37° C. in Lennox lysogeny broth (LB) to an OD600 of 0.6 and induced at 16° C. for 14-18 hours with 1 mM isopropyl B-d-1-thiogalactopyranoside (IPTG) unless otherwise specified. Cells were harvested and purified as follows:

WIP1: In addition to 1 mM IPTG cells were induced with 2 mM MgCl₂. Cell pellets were resuspended in lysis buffer (50 mM Tris-HCl PH7.4, 500 mM NaCl, 10 mM MgCl₂, 10% glycerol, 1 mM dithiothreitol (DTT)) with 1 mM phenylmethylsulphonyl fluoride (PMSF), 1:1000 10 mg/ml (by volume) lysozyme and 1:1000 (by volume) benzonase and were lysed using three passes in a microfluidizer at 10,000 PSI. Cell lysates were cleared by spinning at 16,000 PRM for 45 minutes in an Avanti JA-20 rotor. 10 mM imidazole was added to the cleared lysates. A HisTrap™ HP column on an AKTA FPLC was equilibrated with lysis buffer and 6% elution buffer (50 mM Tris-HCl PH7.4, 500 mM NaCl, 10 mM MgCl₂, 10% glycerol (v/v), 1 mM dithiothreitol (DTT), 400 mM imidazole). Cleared lysates were then run over a HisTrap™ HP, washed with 6% elution buffer for 10 column volumes, and eluted over a gradient to 100% elution buffer over 20 column volumes. Fractions were analyzed using a 10% Tris-Tricine polyacrylamide gel stained with Coomassie brilliant blue solution. Protein containing fractions were pooled and the SUM O-His tags were cleaved with ULP1 protease in dialysis to lysis buffer overnight at 4° C. WIP1 was further purified on a Superdex® 200 16/600 column equilibrated with lysis buffer. Fractions were pooled, concentrated to 200 UM and treated with a 5-fold molar excess of EDTA to remove metal. Chelated WIP1 was buffer exchanged into storage buffer (50 mM Tris-HCl PH7.4, 500 mM NaCl, 10% glycerol (v/v)), flash-frozen and stored at −80° C.

PPM1A, p38 and p38 mutants: Cell pellets were resuspended in lysis buffer (50 mM K*HEPES pH7.5, 200 mM NaCl, 20 mM imidazole, 10% glycerol, 0.5 mM dithiothreitol (DTT)) with 1 mM phenylmethylsulphonyl fluoride (PMSF) and were lysed using three passes in a microfluidizer at 10,000 PSI. Cell lysates were cleared by spinning at 16,000 RPM for 45 minutes in an A vanti JA-20 rotor. A HisTrap™ HP column on an AKTA FPLC was equilibrated with lysis buffer. Cleared lysates were then run over a HisTrap™ HP, washed with lysis buffer buffer for 10 column volumes, and eluted over a gradient to 100% elution buffer elution buffer (50 mM K*HEPES pH7.5, 200 mM NaCl, 400 mM imidazole, 10% glycerol, 0.5 mM dithiothreitol (DTT)) over 20 column volumes. Fractions were analyzed using a 10% Tris-Tricine polyacrylamide gel stained with Coomassie brilliant blue solution. Protein containing fractions were pooled and the 6-His tags were cleaved with 3C protease in dialysis to lysis buffer overnight at 4° C. Cleaved tags were subtracted by passing over a column containing equilibrated Ni-NTA resin. Proteins were further purified on a Superdex200 16/600 column equilibrated with lysis buffer. Fractions were pooled, concentrated to 500 μM, flash-frozen and stored at −80° C.

DUSP3: Cell pellets were resuspended in lysis buffer (50 mM K*HEPES pH 7.5, 200 mM NaCl, 20 mM imidazole, 10% glycerol, 0.5 mM dithiothreitol (DTT)) with 1 mM phenylmethylsulphonyl fluoride (PMSF) and were lysed using three passes in a microfluidizer at 10,000 PSI. Cell lysates were cleared by spinning at 16,000 PRM for 45 minutes in an A vanti JA-20 rotor. A HisTrap™ HP column on an AKTA FPLC was equilibrated with lysis buffer. Cleared lysates were then run over a HisTrap™ HP, washed with lysis buffer for 10 column volumes, and eluted over a gradient to 100% elution buffer elution buffer (50 mM K*HEPES pH7.5, 200 mM NaCl, 400 mM imidazole, 10% glycerol, 0.5 mM dithiothreitol (DTT)) over 20 column volumes. Fractions containing protein were pooled, concentrated to 800 μM, flash-frozen and stored at −80° C.

MKK6^{EE}: Cell pellets were resuspended in lysis buffer (50 mM K*HEPES pH 7.5, 200 mM NaCl, 20 mM imidazole, 10% glycerol, 0.5 mM dithiothreitol (DTT)) with 1 mM phenylmethylsulphonyl fluoride (PMSF) and were lysed using three passes in a microfluidizer at 10,000 PSI. Cell lysates were cleared by spinning at 16,000 PRM for 45 minutes in an A vanti JA-20 rotor. A HisTrap™ HP column on an AKTA FPLC was equilibrated with lysis buffer. Cleared lysates were then run over a HisTrap™ HP, washed with lysis buffer for 10 column volumes, and eluted over a gradient to 100% elution buffer elution buffer (50 mM K*HEPES pH7.5, 200 mM NaCl, 400 mM imidazole, 10% glycerol, 0.5 mM dithiothreitol (DTT)) over 20 column volumes. Fractions were analyzed using a 10% Tris-Tricine polyacrylamide gel stained with Coomassie brilliant blue solution. Protein containing fractions were pooled and were further purified on a Superdex200 16/600 column equilibrated with lysis buffer. Fractions were pooled, concentrated to 500 M, flash-frozen and stored at −80° C.

Crystallographic Methods: p38 was dually phosphorylated using established methods, specifically incubating the constitutively active MKK6S207E/S211E MAP kinase enzyme with p38 at a 1:40 molar ratio. Dual phosphorylation was verified using mass spectrometry.

Crystals of p38-2p apo were obtained by combining 0.3 μL of 8 mg/mL p38-2p in a 0.3 μL reservoir of 100 mM BIS-TRIS pH6.5 and 25% polyethylene glycol (PEG) 3350. Crystals were grown at 20° C. by sitting drop for 2 weeks. Crystals were harvested and flash-frozen in glycerol.

Crystals of p38-2p bound to pexmetinib were obtained by combining 0.3 μL of 8 mg/mL p38-2p+250 μM pexmetinib for a final DMSO concentration of 5% in a 0.3 μL reservoir of 100 mM MES pH6.5, 200 mM ammonium sulfate, 4% propanediol, and 30% PEG8000. Crystals were grown at 20° C. by sitting drop for 2 weeks. Crystals were harvested and flash-frozen in glycerol.

Crystals of p38-2p bound to nilotinib were obtained by combining 0.3 μL of 8 mg/mL p38-2p+250 μMnilotinib for a final DMSO concentration of 5% in a 0.3 μL reservoir of 100 mM BIS-TRIS pH6.0 and 23% PEG3350. Crystals were grown at 20° C. by sitting drop for 2 months. Crystals were harvested and flash-frozen in glycerol.

Crystals of p38-2p bound to BIRB 796 were obtained by combining 0.3 μL of 8 mg/mL p38-2p+250 UM BIRB 796 for a final DMSO concentration of 5% in a 0.3 L reservoir of 100 mM BIS-TRIS pH5.5, 200 mM ammonium sulfate, and 25% PEG3350 for 2 weeks. Crystals were grown at 20° C. by sitting drop. Crystals were harvested and flash-frozen in glycerol.

Crystals of p38 bound to pexmetinib were obtained by combining 0.3 μL of 8 mg/mL p38+250 μM pexmetinib for a final DMSO concentration of 5% in a 0.3 μL reservoir of 100 mM MES pH6.0, 200 mM ammonium sulfate, 4% propanediol, and 20% PEG6000. Crystals were grown at 20° C. by sitting drop for 2 weeks. Crystals were harvested and flash-frozen in glycerol.

Phosphatase assays: All phosphatase assays were performed with p38-2P that was labeled with ³²P by incubating p38 (25 μM), 6His-MKK6^{EE} (0.625 μM), and 20 μCi of γ-³²P ATP for 6-8 hours at room temperature in 20 mM K·HEPES pH7.5, 0.5 mMEDTA, 20 mM MgCl₂, 2 mM DTT. Following initial incubation, excess cold ATP was added for a final concentration of 12 mM and was incubated overnight. Unincorporated nucleotide was removed by buffer exchange using a Zeba™ spin column equilibrated in 50 mM K·HEPES pH7.5, 100 mM NaCl. 6His-MKK6^{EE} was then removed by Ni-NTA resin equilibrated in 50 mM K·HEPES pH7.5, 100 mM NaCl, 20 mM imidazole. The flow-through fraction from the Ni-NTA resin containing p38-³²P was then exchanged into 50 mM K·HEPES pH7.5, 100 mM NaCl buffer using 3 subsequent Zeba spin columns to remove all unincorporated nucleotide and free phosphate. Labeled p38-³²P was aliquoted and frozen at −80° C. for future use.

WIP1 and PPM1A: All WIP1 phosphatase assays were performed at room temperature in 50 mM K HEPES pH7.5, 0.8 mM CHAPS, 0.05 mg/mL BSA, and 15 mM MnCl₂. WIP1 concentration was 2.5 μM, PPM1A concentration was 0.5 μM and p38-³²P was 0.25 μM unless otherwise stated. 1.25 μM p38 inhibitors were added to reactions at a 5% final DMSO concentration immediately before the start of the reaction. Reactions were stopped with 0.5 MEDTA, pH8.0 and run on PEI-Cellulose TLC plates developed in 1 M LiCl₂ and 0.8 M acetic acid and imaged on a Typhoon™ scanner. Phosphatase assays were performed more than three independent times as separate experiments. Data shown in figures is from a single representative experiment, and reported errors are the error from the fit unless indicated otherwise.

DUSP3: All DUSP3 phosphatase assays were performed in 50 mM K HEPES pH7.5 and 100 mM NaCl. Reactions were stopped with SDS and run on PEI-Cellulose TLC plates run through water then developed in 1 M LiCl₂ and 0.8 M acetic acid and imaged on a Typhoon scanner. Phosphatase assays were performed more than three independent times as separate experiments. Data shown in figures is from a single representative experiment, and reported errors are the error from the fit unless indicated otherwise.

+WT p38-³²P: Reactions were run at 37° C. with 50 μM DUSP3 and 0.25 μM p38-³²P. 1.25 μM p38 inhibitors were added to reactions at a 5% final DMSO concentration immediately before the start of the reaction.

+p38^{T180A_32}P: Reactions were run at room temperature with 15 μM DUSP3 and 0.25 μM p38^{T180A_32}. 1.25 μM p38 inhibitors were added to reactions at a 5% final DMSO concentration immediately before the start of the reaction.

SAP: All SAP phosphatase assays were performed at 37° C. in 1× SAP reaction buffer. SAP concentration was 1 unit per 5 μL reaction and p38-³²P concentration was 0.25 μM. 1.25 μM p38 inhibitors were added to reactions at a 5% final DMSO concentration immediately before the start of the reaction. Reactions were stopped with SDS and run on PEI-Cellulose TLC plates run through water then developed in 1 M LiCl₂ and 0.8 M acetic acid and imaged on a Typhoon™ scanner. Phosphatase assays were performed more than three independent times as separate experiments. Data shown in figures is from a single representative experiment, and reported errors are the error from the fit unless indicated otherwise.

Fluorescein diphosphate phosphatase assay: All FDP reactions were performed at 25° C. in a Corning 3573 384-well black flat bottom plate in 50 mM K HEPES pH7.5, 0.8 mM CHAPS, 0.05 mg/mL BSA, 10 mM MnCl₂, 30 nM WIP1 and 50 M FDP. 1.25 μM p38 inhibitors were added at a final DMSO concentration of 5% immediately before the start of the reaction. Fluorescence was read on a plate reader reading from 470 nm to 530 nm every 30 seconds.

Kinase assay: All kinase assays were performed at room temperature in 20 mM K HEPES PH7.5, 0.5 mMEDTA, 20 mM MgCl₂ and 2 mM DTT. 0.5 M MKK6^{EE} was added to 25 μM p38, 25 μM cold ATP and 1 μCi of γ-³²P ATP. 50 μM p38 inhibitors were added to a final DMSO concentration of 5%. Reactions were stopped with SDS and run on PEI-Cellulose TLC plates run through water then developed in 1 M LiCl₂ and 0.8 M acetic acid and imaged on a Typhoon™ scanner.

Cell culture, reagents and antibodies: HL60 cells were maintained in RPM I media supplemented with 2 mM L-glutamine and 10% fetal bovine serum (FBS) at 37° C. in a humidified atmosphere of 5% CO₂. HL60-APPM1A were generated. The following antibodies were obtained from Cell Signaling Technologies (Danvers, MA); PPM1A (cat. #3549), p38 (cat. #9212) and 2p-p38 (cat. #4511). The following antibodies were obtained from A B Clonal (Woburn, MA); p38-phospho-Y182 (cat. #AP0057) and p38-phospho-T180 (cat. #AP0056). B-actin antibody (cat. #sc-47778) was obtained from Santa Cruz Biotechnology (Santa Cruz, CA). Recombinant human TNFα (cat. #300-01A) was obtained from Peprotech (Cranbury, NJ). Lipopolysaccharide (LPS) 500× solution (cat. #50-112-9325) was obtained from Invitrogen (Waltham, MA).

Cellular assays: HL60 cells were cultured in 6 well plates and grown until a density of 2 million cells/mL. Cells were treated with either 40 ng/μL TNFα, 1× LPS, or indicated inhibitor concentrations in a final DMSO concentration of 5%, in the order of addition and time of incubation as indicated. Cells were spun down at 500 xg for 5 minutes, carefully washed with PBS once, and lysed with 50 μL RIPA buffer supplemented with 1:1000 phosphatase inhibitor and 1:500 benzonase. Lysates were centrifuged at 10,000 rpm for 15 minutes. Supernatant was harvested and lysate was either analyzed immediately or flash-frozen and stored at −80° C.

Western blot: Protein concentration of the lysate was determined using Bradford reagent normalized to a standard curve. 40-50 μg total protein per sample was separated on a 10% Tris-tricine polyacrylamide gel and then transferred to a polyvinylidene (PV DF) membrane using the Bio-Rad transfer system. Standard blotting protocol was followed. Primary antibodies (see antibody section) were incubated overnight at 4° C. followed by near-infrared (NIR) labeled secondary antibodies Goat anti-Rabbit IgG (H+L) Cross-Adsorbed Secondary Antibody, DyLight™ 800 (cat #. SA5-10036) or IgG (H+L) Cross-Adsorbed Goat anti-mouse, DyLight™ 680, Invitrogen™ (cat #. 35519) obtained from Invitrogen (Waltham, MA) for 1 hour at room temperature in 2.5% milk in TBST. Blots were washed and imaged on a LI-COR imaging instrument.

IC50 measurement: Cellular IC50 was measured using the Lumit™ TNF-α Human Immunoassay (cat. #6050) from Promega (Madison, WI). HL60 cells were concentrated to 5 million cells/mL and varying concentrations of p38 inhibitors as indicated were used, maintaining a final DMSO concentration of 2%. Assay was completed following manufacturers protocol.

PPM1A Knockout (sgR NA sequences taken from Berton et al, 2022)

Cloning of Lentiviral Plasmid: The Crispr/Cas9 knock-out plasmids targeting PPM1A were created by ligating annealed oligonucleotides containing single guide RNA (sgRNA) sequences into the digested LentiCRISRv2 blast vector, as previously described.

Production of lentivirus: HEK293T cells were seeded at 50% confluency and transfected 24 hours later with three plasmids; LentiCRISPR v2 blast constructs with psPA×2 and pM D2.G. Lentivirus media was collected at 48 and 72 hours, centrifuged, sterile filtered and stored for less than 1 week at 4° C.

Lentiviral infection and generation of knockout line: Wild type HL60 cells underwent lentiviral infection in the presence of 8 μg/ml polybrene. After 24 hours the lentiviral media was removed and replaced with conditioned media. After 72 hours of recovery in conditioned media the selection process began by treating cells with 10 μg/mL blasticidin and incubated for 10 days, supplementing with fresh blasticidin on day 5, leaving the media unchanged. Cells were serially diluted into a 96 well plate with conditioned media and 7 μg/mL blasticidin. They were monitored, wells with cell growth were expanded and then analysed by western blot and Sanger sequencing to verify PPM1A knockout.

SEQ ID NO: 1
5′TGCCAAGTGGACTTGAATCG guide 3 - source of KO,
SEQ ID NO: 2
5′AGAAGTGGGTCAACAGCTGT guide 1,
SEQ ID NO: 3
5′ TCACAAACCAAGTAATCCGC guide 2,

### Example 1: Type-II p38 MAP Kinase Inhibitors Direct Threonine Dephosphorylation

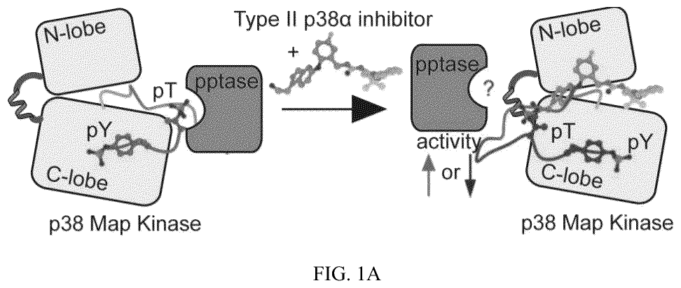

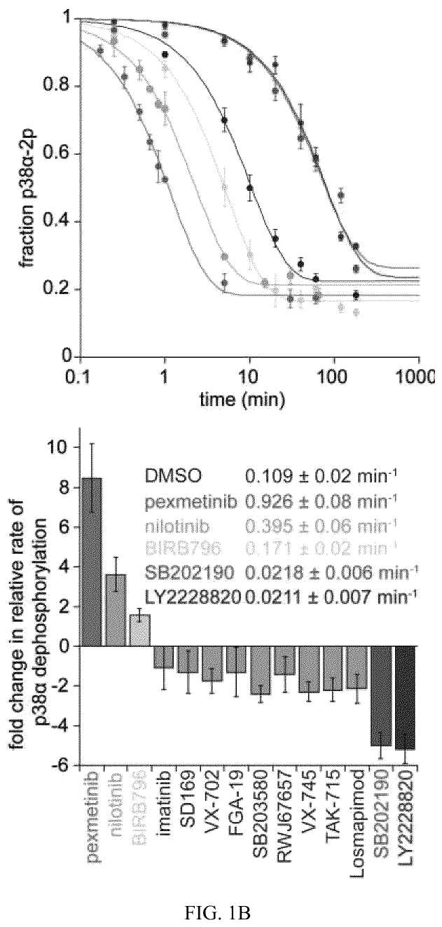

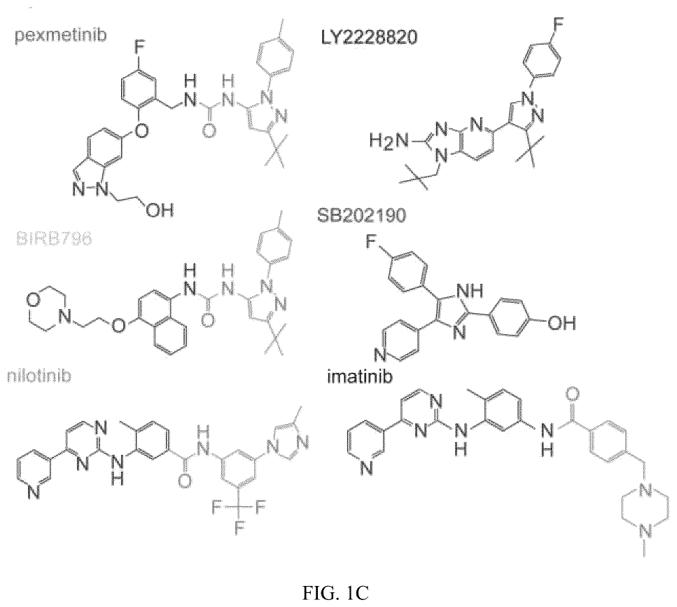

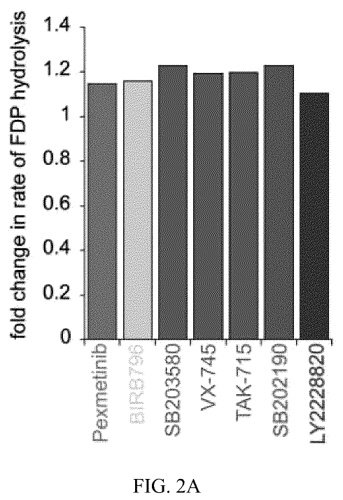

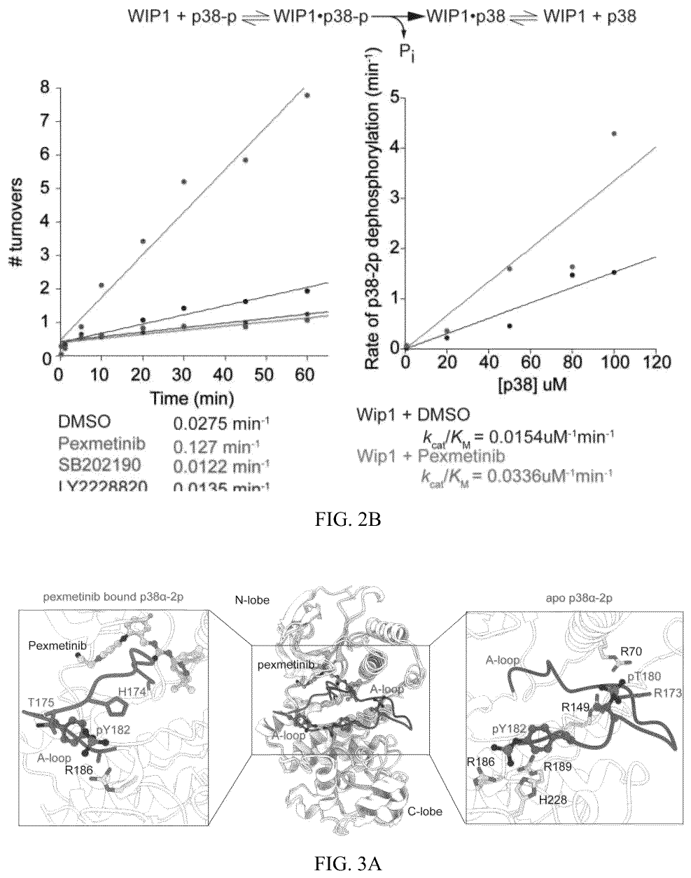

To determine whether the conformation of the p38α activation loop impacts its dephosphorylation by WIP1, WIP1 phosphatase activity towards p38α bound to a panel of type II inhibitors was measured (FIG. 1A). From an initial panel of thirteen inhibitors, pexmetinib and BIR B 796 increased the rate of threonine dephosphorylation (by 8 and 2-fold respectively), and that LY2228820 and SB202190 decreased the rate of dephosphorylation (by 5-fold) (FIG. 1B). Pexmetinib and BIRB 796, share an identical right-side that projects into an allosteric pocket that displaces the activation loop (based on the structure of BIR B 796 bound to unphosphorylated p38α, PDB: 1K V2), but differ in their left-sides that bind in the ATP binding site (FIG. 1C). In contrast, SB202190 and LY2228820 lack a right-side element and the structural changes to p38α are unknown. Confirming that the changes in dephosphorylation were caused by inhibitor interaction with p38α, the inhibitors did not change WIP1 hydrolysis of fluorescein-diphosphate, which does not interact with the compounds (FIG. 2A). Although the high K_{M} of WIP1 for p38α prevented us from determining whether the compounds change the k_{cat} or K_{M} of the reaction, we observed similar effects under single or multiple turnover conditions (FIG. 2B-C). Together, these results demonstrate that type II p38α inhibitors can modulate the rate of dephosphorylation.

### Example 2: Pexmetinib Presents Phospho-Threonine for Dephosphorylation

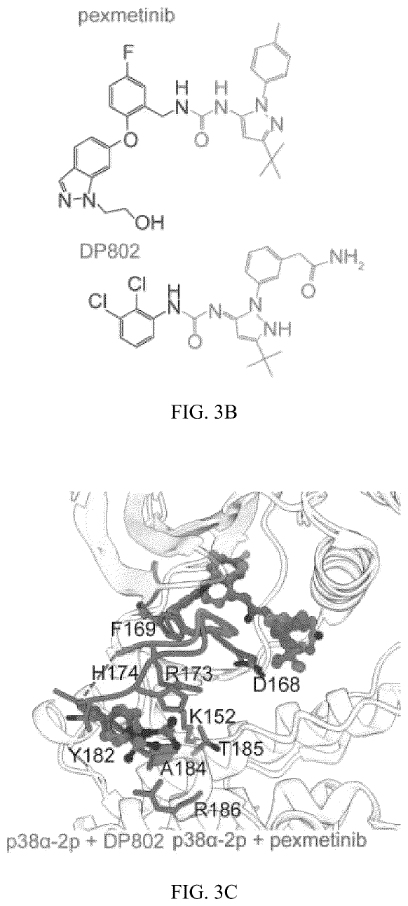

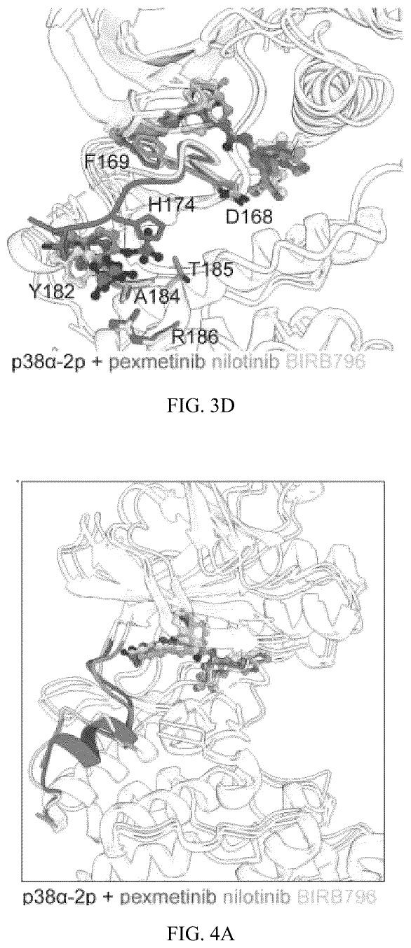

To determine how phosphatase activity is controlled, an X-ray crystal structure of dual-phosphorylated p38 bound to pexmetinib was solved (FIG. 3A). Pexmetinib flipped the activation-loop to dock near the ATP binding pocket, when compared to an unliganded structure of dual-phosphorylated human p38α that we determined (FIG. 3A). Displacement of the activation loop from the unliganded conformation is driven by clashes of the right-side of pexmetinib and hydrogen bonding of the carbonyl oxygen of pexmetinib to the nitrogen of F169 of DFG. In the flipped conformation, R 186 rotates with the phospho-tyrosine to form a docking site where the phospho-tyrosine additionally interacts with H174 and T175. This links the N- and C-terminal ends of the activation loop and projects the phospho-threonine to the solvent (phospho-threonine 180 and the four preceding amino acids are not resolved in the density maps) (FIG. 3A, left zoom). Without being held to theory, it is hypothesized that this conformational change presents the phospho-threonine for dephosphorylation by WIP1.

### Example 3: a Shared Conformational State Directs p38α Dephosphorylation

To determine if any of the hundreds of structures of p38 in the PDB share the activation-loop conformation of pexmetinib bound dual-phosphorylated p38α, a DALI search was performed. This revealed a structure of dual-phosphorylated p38α bound to DP802, a compound with similar right-side and shorter left-side compared to pexmetinib (FIG. 3B, PDB:3NNX,). The portion of the activation-loop that is ordered places phospho-tyrosine in a similar position as in our pexmetinib bound structure (FIG. 3C). Although DP802 was not commercially available for phosphatase assays, this structure supports our hypothesis that the right side of pexmetinib is critical for activation-loop flipping and predicts that DP802 would similarly stimulate p38α dephosphorylation.

A search of deposited kinase structures using KinCoRe revealed one additional structure with a similar activation loop conformation: unphosphorylated p38ß bound to the ABL inhibitor nilotinib (Tasigna, PDB: 3GP0, p38B has 73.6% identity to p38α). This structure has DFG and the activation-loop tyrosine in remarkably similar conformation to our pexmetinib bound structure. Consistent with the prediction that the pexmetinib induced activation-loop conformation presents the phospho-threonine for dephosphorylation, nilotinib stimulated WIP1 dephosphorylation of p38α four-fold (FIG. 1B). Crystal structures of dual-phosphorylated p38α bound to nilotinib and BIR B 796 revealed that both compounds induce the flipped activation loop conformation initially observed with pexmetinib (FIG. 2D). The region of the activation loop that is resolved in the electron density of our three structures is slightly different (unmodeled residues: pexmetinib 176-180, nilotinib 170-181, and BIRB 796 174-180), but does not correlate with the magnitude of phosphatase stimulation. The shared activation-loop flip, DFG conformation, and phospho-tyrosine position of these four structures confirms that the compounds dictate activation-loop rearrangement rather than crystal contacts or interactions with crystallization compounds. Intriguingly, imatinib (Gleevec®, bound to unphosphorylated p38α PDB: 3HEC), which shares a left-side with nilotinib projects into the space occupied by the activation-loop of phosphorylated p38α and induces a similar DFG out conformation. However, imatinib did not stimulate dephosphorylation of p38α, suggesting that the DFG conformation and activation-loop displacement is necessary but not sufficient to present the phospho-threonine for WIP1 (FIG. 1B).

This raises the question of what features of the type II inhibitors determine the magnitude of WIP1 stimulation. Two well established allosteric networks in p38α show conformational changes that correlate with the magnitude of WIP1 stimulation:

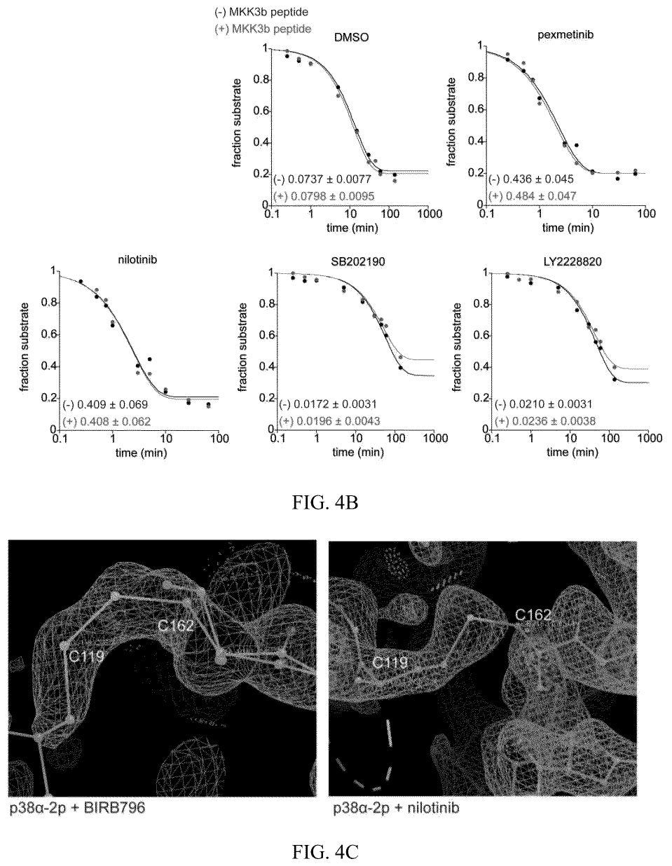

1) The αD helix: The αD helix, which forms the hinge between the N- and C-lobes of p38α is progressively unwound in our dual-phosphorylated nilotinib and BIR B 796 bound structures (FIG. 4A). The left side of the compounds make contacts above the αD helix, which is positioned directly behind the flipped activation-loop, suggesting a possible mechanism for stabilizing the activation loop flipped conformation. Notably, the αD helix is a known allosteric docking site for kinase interacting motif peptides (KIM) to p38α and other MAPKinases, although we did we did not detect any effect of KIM peptide (from MKK3B) on WIP1 dephosphorylation (FIG. 4B). One other feature of the αD helix in our structures is that a disulfide bond is formed between C119 of the αD helix and C162 in nilotinib and BIRB 796 bound structures that was recently found to inhibit p38α binding to KIM peptides (FIG. 4C).

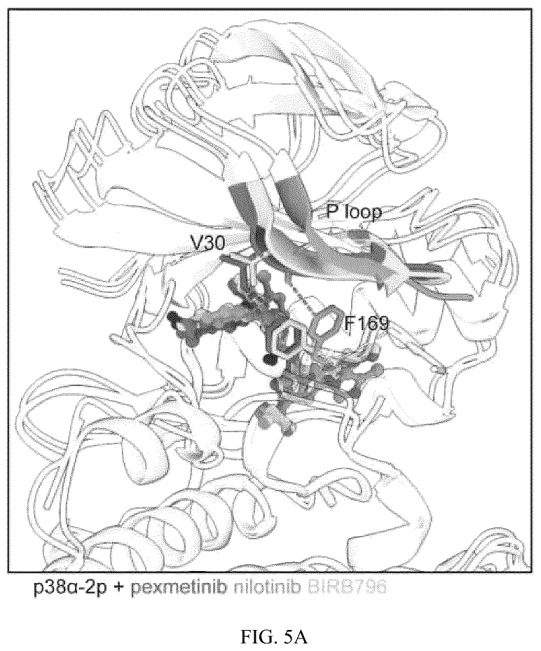

2) The N-lobe and P-loop: The P-loop sits above the ATP binding site of kinases and closes to promote catalysis. The P-loop is progressively more closed in the BIRB 796, nilotinib, and pexmetinib bound structures of dual-phosphorylated p38α (FIG. 5A). This makes contacts with F169 of the DFG motif, potentially stabilizing the flipped activation loop conformation. This closure is correlated with an overall downward motion of the N-lobe that is caused by interaction of V30 with the left-sides of the type II inhibitors, suggesting a possible link to the compound structure (FIG. 5A). Of additional note, comparison of the two chains of unliganded dual-phosphorylated human p38α that we solved and the previous structure of dual-phosphorylated mouse p38α (PDB: 3PY3, there are only two residues difference in sequence between human and mouse p38α, but both are involved in crystal contacts in the mouse structure) revealed different degrees of rotation of this region between the three chains (FIG. 5B). These structures provide a model for how N-lobe conformation fluctuates in the absence of ligand and is stabilized by ligand binding, consistent with previous NM R studies of p38α. These structures, however, contrast with recent M D simulations starting with the mouse p38α model in that the degree of conformational heterogeneity observed between X-ray structures is much smaller.

### Example 4: Phospho-Tyrosine is not Required to Stimulate Threonine-Dephosphorylation

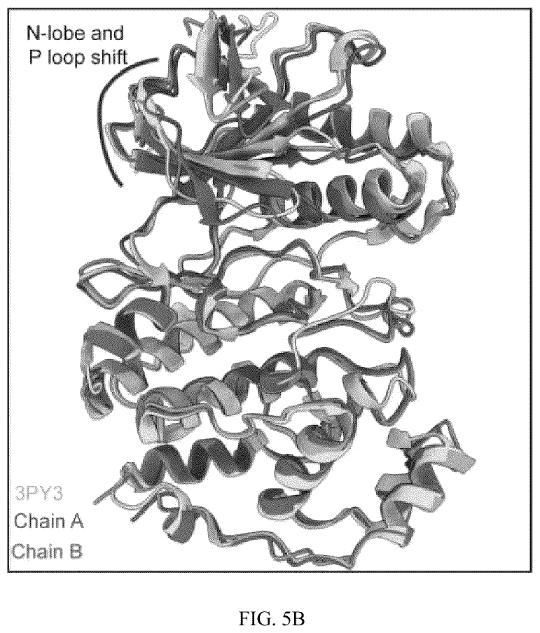

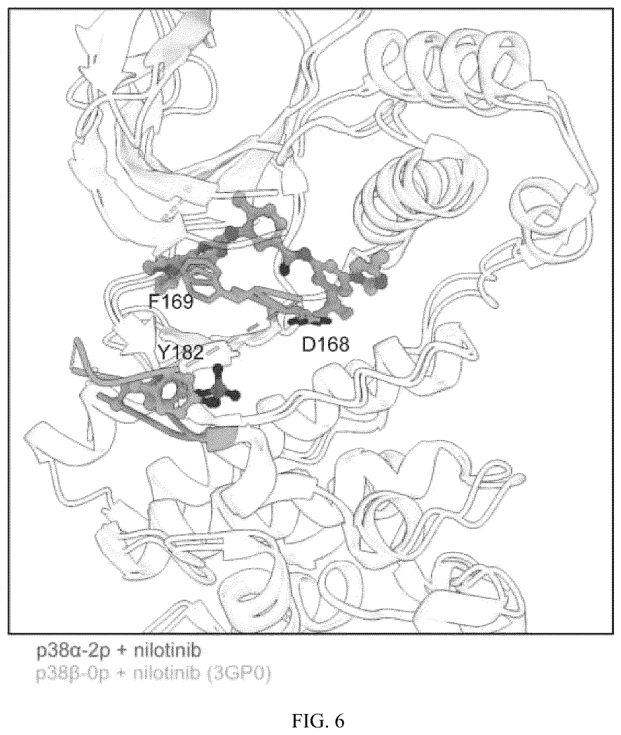

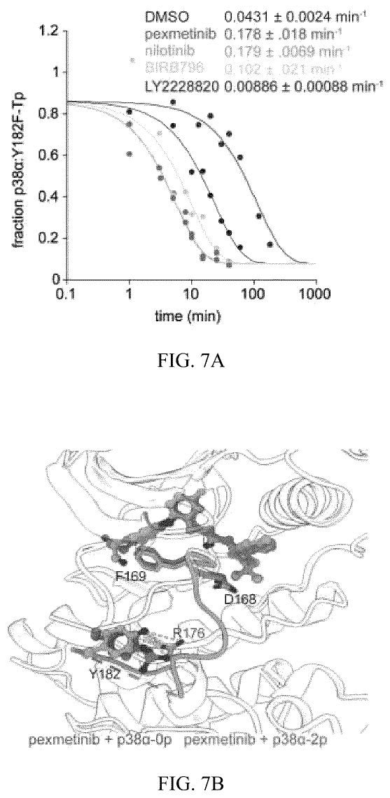

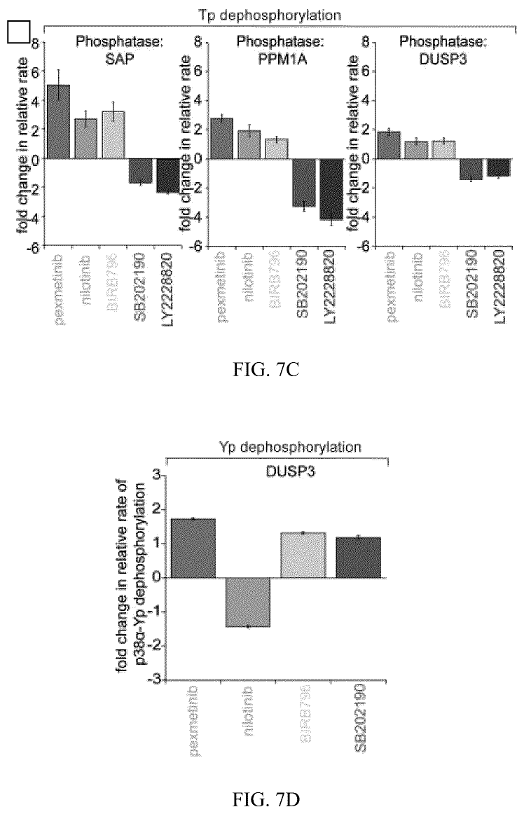

While the activation-loop in all the inhibitor-bound structures appears to be anchored by the phospho-tyrosine, the flipped activation loop conformation was preserved in the structure of nilotinib bound to unphosphorylated p38ß (FIG. 6). It was then tested whether tyrosine-phosphorylation is required to stimulate WIP1 dephosphorylation using singly-threonine phosphorylated p38α (with tyrosine 180 substituted with phenylalanine, p38@Y182F). WIP1 dephosphorylation of p38qY182F was indistinguishable from p38α and was similarly increased by the type II inhibitors with the exception that the magnitude of pexmetinib stimulation was reduced to four-fold (FIG. 7A). An X-ray crystal structure of unphosphorylated p38α bound to pexmetinib was solved to determine whether tyrosine phosphorylation was required for activation-loop flipping. Consistent with the biochemical results, the unphosphorylated activation-loop is flipped and tyrosine is similarly positioned as in the dual-phosphorylated, pexmetinib bound structure (FIG. 7B). The tyrosine is not as well coordinated as in the dual-phosphorylated structure but is similarly anchored from the N-terminal side of the activation loop, now coordinated by a new contact from D176 (FIG. 7B). The resolution of the structure is significantly lower, indicative of an overall increase in conformational flexibility, and consistent with reduced WIP1 stimulation. Together, it was concluded that tyrosine phosphorylation promotes, but is not required for pexmetinib induced presentation of the p38α phospho-threonine for dephosphorylation by WIP1.

### Example 5: Conformational Selectivity of Activation Loop Recognition is Shared Across Phosphatase Families

Because displacement of the phospho-threonine is a shared feature of the type II inhibitors that present p38α for WIP1 dephosphorylation, it was reasoned that other phosphatases might similarly be stimulated. The subset of inhibitors that had the largest effects was surveyed with three additional phosphatases: PPM1A, a PPM phosphatase related to WIP1 that natively targets p38, DUSP3, a dual specificity family phosphatase (related to tyrosine phosphatases) that is capable of dephosphorylating p38α and has been well characterized biochemically, and alkaline phosphatase from shrimp (SAP), which exhibits minimal specificity and can dephosphorylate both phospho-threonine and tyrosine. While none of these phosphatases were affected to the extent of WIP1, the same trends of stimulation and inhibition were observed (FIG. 7C). SAP had the largest change in activity followed by PPM1A and DUSP3. To determine if this effect was phosphorylation site specific, we assayed DUSP3 dephosphorylation of single tyrosine phosphorylated p38α (T180A) in the presence of pexmetinib, nilotinib, BIRB 796 and SB202190 and found no effect on dephosphorylation rate (FIG. 7D). Thus, the effect of type II inhibitors on p38α threonine dephosphorylation is generalizable across phosphatase families, but the magnitude of change is phosphatase specific.

### Example 6: Type II Inhibitors Control p38α Dephosphorylation in Human Cells

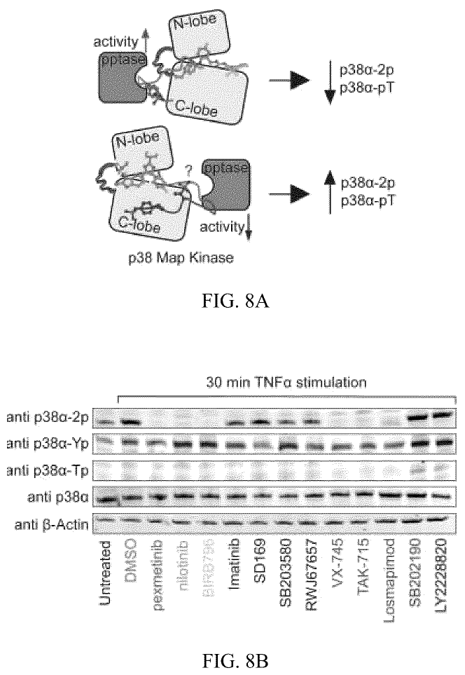

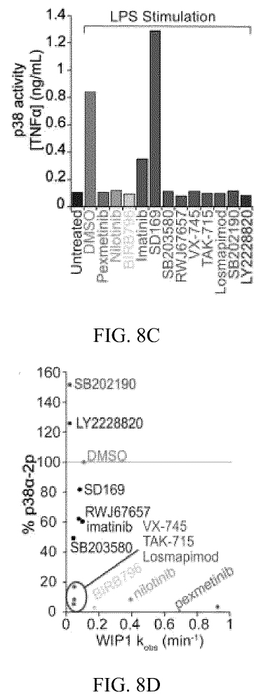

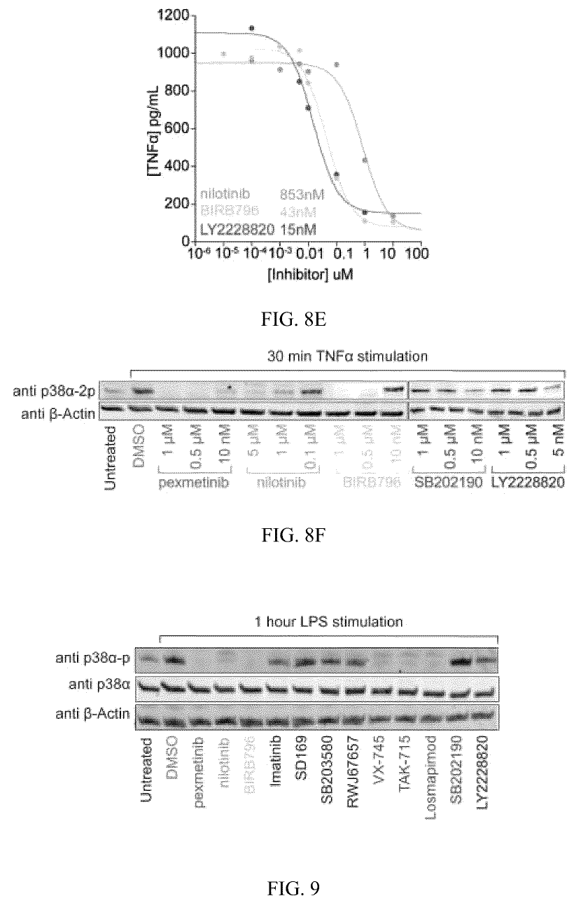

Without being held to theory, it was hypothesized that the cellular mechanism of action of type II p38α inhibitors is modified by phosphatase mediated changes in p38α phosphorylation (FIG. 8A). To test this hypothesis, the level of dual-phosphorylated p38α in human HL60 cells treated with the panel of p38 inhibitors after the initiation of the inflammatory response with TNFα or LPS was determined (FIGS. 8B and 9). p38α phosphorylation showed a remarkable correlation with our biochemical results: dual-phosphorylated p38α disappeared in cells treated with pexmetinib, nilotinib, and BIR B 796 and increased in cells treated with SB202190 and LY2228820 (FIG. 8C). Importantly, total p38α levels were unchanged and p38α downstream signaling was inhibited regardless of p38α phosphorylation status (the one exception is SD-169, which had no change on phosphatase activity FIGS. 8B-C). Further supporting this correlation, DP802 treatment caused disappearance of dual-phosphorylated p38α in HELA cells. Three compounds (VX-745, TAK-715 and losmapimod) that did not affect WIP1 threonine dephosphorylation of p38α in biochemical assays, nonetheless caused disappearance of dual-phosphorylated p38α in HL60 cells (FIGS. 1B and 8B), which therefore appears to be caused by a distinct mechanism.

Several lines of evidence suggest that cellular phosphatases cause the inhibitor-dependent changes in p38α phosphorylation observed in HL60 cells (WIP1 is not expressed in these cells and PPM1A deletion did not restore p38α phosphorylation following compound treatment (FIG. 9)). First, phospho-site specific antibodies revealed disappearance of threonine phosphorylation and increase of single-tyrosine phosphorylated p38α in cells treated with pexmetinib, nilotinib and BIRB 796 (FIG. 8B). Reciprocally, cells treated with SB202190 had increased threonine phosphorylation and cells treated with VX745, TAK715, and losmapimod did not accumulate phospho-tyrosine (FIG. 8B). These results are all consistent with pexmetinib, nilotinib, and BIR B 796 acting through threonine phosphatases while, VX745, TAK715, and losmapimod decrease dual-phosphorylated p38α by an alternative mechanism. Second, p38α threonine phosphorylation completely disappeared when cells pretreated with TNFα were harvested immediately after addition of pexmetinib, nilotinib, and BIRB 796, suggestive of rapid phosphatase action on pre-phosphorylated p38α. Third, pexmetinib, nilotinib, and BIRB 796 did not impact threonine phosphorylation of p38α by MKK6 in biochemical assays. This notably contrasts with previous reports that BIR B 796 and some other type II inhibitors block MKK6 dual phosphorylation measured by ELISA. Fourth, the concentration dependence of p38α inhibition is similar to the concentration dependence of p38α threonine dephosphorylation, suggesting p38α dephosphorylation is caused by inhibitor binding to p38α (FIGS. 4E-F). Thus, it is concluded that phosphatase modulating type II kinase inhibitors have a second mechanism of action in cells, driving dephosphorylation of p38α, in addition to direct inhibition.

### DISCUSSION

These findings raise the question of whether the conformation of p38α trapped by phosphatase stimulating inhibitors reflects the native mechanism to control p38α dephosphorylation. Several lines of evidence suggest that this may be possible. First, the unliganded structure of SnRK2.6 kinase bound to HA B 1 phosphatase revealed the kinase activation-loop in an inactive conformation, the P-loop closed down, and repositioned αD helix similar to the conformation of p38α that we found stimulates dephosphorylation. Importantly, the disordered region of the p38α structures would allow the phospho-threonine to access the position of the phosphorylated serine of SnRK2.6 in the phosphatase bound structure. Remarkably, the activation-loop of p38α assumed a similar orientation when bound to its activating kinase MKK6, and the region of interaction is very similar to that of SnRK2.6 and HAB1. This raises the possibility that the flipped activation-loop conformation of p38α trapped by pexmetinib, nilotinib, and BIRB 796 may be a shared modification-competent state for MAPKinases and phosphatases. In contrast, inhibitors that trap the activation-loop of the MAPKinase ERK in an inactive conformation (distinct from the flipped-p38α conformation we observe) have been found to stimulate ERK tyrosine dephosphorylation. This is consistent with our finding that the kinase activation loop poses recognized for dephosphorylation are phospho-site specific and further suggests that they are kinase specific. Together, these findings indicate that type II kinase inhibitors may achieve high-affinity and specificity by trapping states that evolved for phosphatase recognition.

It is hypothesized that stimulating dephosphorylation and directly competing for ATP binding enhance the potency and/or specificity of kinase inhibition. A type II kinase inhibitor such as pexmetinib, nilotinib, or BIR B 796 that stimulates dephosphorylation of its target could achieve increased efficacy through durable inactivation even after disassociating, a conceptually similar mechanism to how PROTAC targeted protein degrading drugs bind and then destroy their targets. In contrast, compounds such as SB202190 and LY2228820 that inhibit dephosphorylation could increase the pool of active kinase that requires inhibition reducing potency. The cell-type variability of phosphatase activity thus raises the possibility that this dual-action inhibition mechanism could enable rationally guided identification of cell-type specific kinase inhibitors.

The finding that nilotinib stimulates p38α dephosphorylation may provide an example of how phosphatase interaction with type II inhibitors has clinical relevance. Initial biochemical kinase screening identified p38 as a weak off-target binder of nilotinib compared to its primary target ABL. However, nilotinib exhibited side effects correlated with p38 inhibition that could be exacerbated by phosphatase interaction. Conversely, nilotinib has been investigated as a treatment for neurodegenerative diseases including Lewy Body Dementias, Alzheimer's disease, and Parkinson's disease, with therapeutic benefit attributed to reduction of inflammation in the brain that could be attributed to p38 inhibition. Similarly, the p38α inhibitor VX-745 (neflamapimod) has shown promise in trials for neurodegenerative diseases with challenges of identifying optimal dosing. Thus, the discovery that type II kinase inhibitors can control phosphatase activity towards their targets could enable development of better drugs, emphasizing the importance of understanding how the conformation of kinases controls their dephosphorylation and revealing a potential benefit of identifying the complement of phosphatases that dephosphorylate kinases to control signaling decisions in diverse cell-types and diseases.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of determining if a kinase inhibitor is a phosphatase enhancer or inhibitor, comprising
providing a phosphorylated target kinase substrate;
contacting the kinase inhibitor, the phosphorylated target kinase substrate and the phosphatase under conditions for dephosphorylation of the target kinase substrate by the phosphatase;
quantitating dephosphorylation of the phosphorylated target kinase substrate in the presence and absence of the kinase inhibitor; and
determining the kinase inhibitor is a phosphatase enhancer when its presence increases the rate of dephosphorylation of the target kinase substrate, or
determining the kinase inhibitor is a phosphatase inhibitor when its presence decreases the rate of dephosphorylation of the target kinase substrate.

2. The method of claim 1, further comprising determining the x-ray crystal structure of the kinase inhibitor bound to the phosphorylated target kinase substrate, and identifying changes in the phosphorylated target kinase substrate in the presence of the bound kinase inhibitor.

3. The method of claim 2, wherein the change in the phosphorylated target kinase substrate in the presence of the bound kinase inhibitor is a change in the activation loop conformation.

4. The method of claim 1, further comprising performing a cellular assay for monitoring the effect of the kinase inhibitor on the target kinase phosphorylation, or a cellular assay for monitoring enhancement/inhibition of the kinase inhibitor on phosphatase activity.

5. The method of claim 1, wherein the kinase inhibitor is an ATP-competitive kinase inhibitor.

6. The method of claim 1, wherein the kinase inhibitor is a type II ATP-competitive kinase inhibitor.

7. The method of claim 1, wherein the kinase inhibitor is an allosteric kinase inhibitor.

8. The method of claim 1, wherein the target kinase is a serine/threonine protein kinase, a tyrosine-specific kinase, a receptor tyrosine kinase, a receptor-associated tyrosine kinase, or a dual-specificity kinase.

9. The method of claim 1, wherein the target kinase is a mitogen-activated protein kinase selected from an extracellular signal-regulated kinase, a c-Jun N-terminal kinase, a p38 isoform, ERK5, ERK3, ERK4 or ERK7/8.

10. The method of claim 1, wherein the target kinase is p38α MAP kinase.

11. The method of claim 10, wherein the phosphatase is WIP1, PPMIA, PTP or a DUSP phosphatase.

12. The method of claim 1, wherein, prior to optimizing, the kinase inhibitor was identified by determining binding affinity to the target kinase and/or determining target kinase inhibition through a high throughput kinase assay measuring the uptake of ATP.

13. The method of claim 1, wherein the phosphorylated target kinase substrate is prepared in vivo in a reaction comprising a cognate upstream kinase and ATP.

14. The method of claim 1, wherein the phosphorylated target kinase substrate is prepared in a cell by initiation of an immune response with TNFα or LPS.

15. The method of claim 1, further comprising administering the kinase inhibitor to a subject in need of such treatment.

16. A multiwell assay, wherein each well comprises a phosphorylated target kinase substrate, a phosphatase, a buffer, and a means for quantitating dephosphorylation of the phosphorylated target kinase substrate.

17. The multiwell assay of claim 16, wherein the means for quantitating dephosphorylation of the phosphorylated target kinase substrate comprises a reagent.

18. The multiwell assay of claim 16, wherein the means for quantitating dephosphorylation of the phosphorylated target kinase substrate comprises a radioactive or fluorescent phosphate probe.


SEQUENCE LISTING (3 sequences)

SEQ ID NO: 1 - RNA, 20 nt, synthetic construct
  source 1..20: mol_type = other RNA
tgccaagtggacttgaatcg

SEQ ID NO: 2 - RNA, 20 nt, synthetic construct
  source 1..20: mol_type = other RNA
agaagtgggtcaacagctgt

SEQ ID NO: 3 - RNA, 20 nt, synthetic construct
  source 1..20: mol_type = other RNA
tcacaaaccaagtaatccgc